(19) United States
(12) Patent Application Publication
(10) Pub. No.: US 2014/0330124 A1
(43) Pub. Date: Nov. 6, 2014

(54) FLEXIBLE ENDOSCOPIC PROBE SYSTEM AND METHOD OF USING SAME

(71) Applicant: SonaCare Medical, LLC, Charlotte, NC (US)

(72) Inventor: Mark Carol, Charlotte, NC (US)

(73) Assignee: SonaCare Medical, LLC, Charlotte, NC (US)

(21) Appl. No.: 14/269,469

(22) Filed: May 5, 2014

Related U.S. Application Data
(60) Provisional application No. 61/818,987, filed on May 3, 2013.

(51) Int. Cl. A61N 7/00; A61B 8/12

(52) U.S. Cl. CPC A61N 7/00; A61B 8/12; A61N 2007/0004
USPC 600/439; 601/2

(57) ABSTRACT

A system for providing ultrasound includes a drive shaft having a proximal end and a distal end. One or more motors are positioned at or near the proximal end of the drive shaft. One or more pair of jaws or one or more joints are mounted on or near the distal end of the drive shaft. One or more transducers are configured to generate thermal or cavitational lesions with ultrasound. Each transducer is mounted to one of the jaws or is operatively connected to the joint.

### RELATED APPLICATION DATA

This application claims priority to U.S. Provisional Patent Application No. 61/818,987, filed May 3, 2013 and entitled “Flexible Endoscope Probe,” which is hereby incorporated by reference in its entirety.

### BACKGROUND

Focused ultrasound devices use ultrasound (“US”) transducers to deliver a generally thermal or cavitational dose to a small, well-defined spot at some fixed distance, focal distance or relative distance from a transducer surface. One or more ultrasound crystals may be combined to form a transducer that can be geometrically or electronically focused at a point distant from the surface of the transducer, thereby concentrating the US energy at the focal spot. Such concentration of sound energy results in cavitational and thermal damage to the region of focus and can be used, among other things, to destroy cancerous tissue.

One way to deliver thermal dose to a larger region is to move the transducer so that the small spot of thermal dose is applied over the region that is to receive thermal or cavitational dose(s). Alternatively, the patient may be moved relative to the transducer. The latter approach is often used in extracorporeal devices where the transducer is located outside the patient; for example, InSightec, Ltd's EXABLATE™ system. The former approach, in comparison, is often used in devices where the transducer is located inside the patient. Such is the case with devices such as the SonCare Medical's SONATHERM™ and SONABLATE™ devices.

In devices where the transducer is introduced into the patient and is moved potentially relative to the patient, it is typically deployed in a probe housing. The probe will typically include a way to couple the transducer to the tissue to be treated. Coupling involves providing a continuous water path between the transducer and the tissue being treated. In addition, the coupling mechanism is used to control the depth of the focal point of the transducer in the region of interest; increasing the depth of the water contained by the means for coupling allows the focal point of the transducer to be moved deeper or shallower in the tissue to which it is coupled. The probe may contain an US transparent window through which the thermal US energy passes. This window typically is larger than the transducer. The transducer can be moved around inside the window in order to deliver dose to a region greater in width and or length than the size of the transducer itself.

Rigid shaft-based drive systems can be employed to move the transducer inside the probe so that that the spot of thermal dose can be scanned over the region that is to receive thermal or cavitational dose without the need to move the probe itself around inside the patient. This reduces the amount of trauma to which the patient would be subjected, and the loss of tissue coupling that would occur, if the probe itself were moved around. However, these types of systems increase the size of the probe due to the need for the window to be as large as the largest volume of tissue to be treated.

Typically, such drive systems, including motors that are connected to the transducer by a shaft, are housed within the probe body that houses the transducer. Motors are provided to move the transducer across multiple axes. Approaches using a rigid straight shaft require a line of sight or a direct path for the probe to the targeted tissue. With such geometry, it may not be possible to position the transducer so that it can reach targets that may lie on the underside or backside of an organ, since the shaft cannot be bent such that it can “see” around a corner or other obstruction. In addition, it may be difficult to deliver a high intensity focused ultrasound (“HIFU”) treatment to regions located directly in front of or behind the axis of the probe if the US window effectively is on the side of the shaft.

The transducers could be mounted at the end of the probe in a forward facing direction. However, because of the length of transducer assemblies used to deliver HIFU and the need to scan these transducers over a region greater than their length, mounting the transducers at the end of a shaft orthogonal to the shaft would necessitate a very large surgical opening in order to introduce the probe into a patient.

Flexible delivery devices for HIFU have been developed. For instance, U.S. Pat. No. 5,492,126 (Hennige) is an example of such a device, which allows the position of the transducer to be adjusted relative to the orientation of its long axis. However the device of Hennige, which consists of a focused ultrasound (“FUS”) transducer incorporated into a flexible endoscope, requires manual positioning of the transducer and is not capable of being scanned over a large region. Another approach can be found in U.S. Pat. No. 7,591,794 (Lacoste), which teaches ways of angulating the end of the probe containing the transducer. However, Lacoste does not teach the use of focused ultrasound. Therefore, Lascoste teaches a relatively inefficient means of delivering ablative energy. Further, Lascoste does not teach the use of integrated US imaging.

### SUMMARY

It would be desirable to provide a means of moving a transducer inside a patient without requiring line of sight, without the need to enlarge the size of the probe to accommodate movement of the transducer within the probe, without the need to utilize a large surgical opening in order to introduce the probe, with the ability to use focused ultrasound for the treatment, and/or with the integration of optical and US imaging. The device, system and method of the present disclosure accomplish the above and other objectives.

According to an embodiment of the present disclosure, an ultrasound probe includes ultrasound transducers capable of generating thermal or cavitational lesions with US and optionally of imaging such lesions with US, attached to one or more pairs of jaws mounted on the end of a flexible drive shaft. The probe may include a tissue coupling mechanism including a fixed or variable fillable fluid membrane with ingress and egress ports secured in a manner that covers the transducers. The flexible shaft can be connected to a set of motors that may be activated manually or under computer control to adjust the position of the transducer assembly. The motors may control all or some combination of: bending of the shaft, opening and closing of the jaws, rotation of the shaft and jaws together, and/or rotation of the jaws themselves. Computer or manually controlled movement of the various degrees of freedom of the system may be provided to allow the probe with the jaws closed to be inserted through a small opening in the patient in order to cause the jaws, and thereby the transducers, to be deployed in an open treatment position once inside the patient, the probe to be positioned correctly relative to the region to be treated, and the focal spot of the transducer scanned over the region to be treated by a combination of controlled movements including bending and rotating.

In a further embodiment of the present disclosure, various portions of the probe and transducers can be equipped with localization technology so that the position and orientation of various portions of the probe and transducer relative to the target and/or a fixed point in space, or relative to a known reference, can be determined.

In another embodiment of the present disclosure, the probe may include an optical imaging system at its distal end, thereby allowing the region that is to be treated to be visualized optically.

In an additional embodiment of the present disclosure, the probe may be deployed in a fluid filled chamber where the probe itself does not require a tissue coupling mechanism and where ingress and egress of fluid into the chamber is controlled through separate means. The volume of the chamber may be used to determine the position of the focal spot in the chamber wall.

In an alternative embodiment of the present disclosure, an ultrasound probe includes ultrasound transducers capable of generating thermal or cavitational lesions with US and optionally of imaging such lesions with US, attached to the end of a flexible drive shaft oriented such that the direction of the therapy and imaging US beams is at least generally, if not exactly, orthogonal to the long axis of the shaft and the position of the transducers relative to the shaft may be adjusted. The probe may include a tissue coupling mechanism including a fixed or variable fillable fluid membrane with ingress and egress ports secured so that covers the transducers. The flexible shaft can be connected to a set of motors that can be activated manually or under computer control to adjust the position of the transducer assembly. The motors can control all or some combination of: bending of the shaft, rotation of the entire shaft, and/or rotation of the end of the shaft only. Computer or manually controlled movement of the various degrees of freedom of the system may be provided to allow the probe to be inserted through a small opening in the patient, then angled to the desired treatment position once inside the patient, the probe to be positioned correctly relative to the region to be treated to be treated by a combination of controlled movements including bending of the shaft and rotation the entire shaft, which will direct the body of the transducer in the correct direction, and rotation of the end of the shaft, which will direct the active portion of the transducer in the desired direction.

In another embodiment of the present disclosure, a method of delivering a FUS treatment is provided. The method may include advancing into a patient through a surgically created or naturally occurring opening a probe that contains at least a single pair of closed jaws each fitted with a FUS transducer, that may contain an imaging transducer mounted at the center of the jaws, and includes a means for coupling the transducer to the tissue to be treated through which is installed an acoustic window; under computer or manual control advancing the probe while using a flexible drive shaft system that can change the shape of the probe so as to adjust the position of the end of the probe and bring it in proximity to the region to be treated; opening the jaws under computer or manual control so that the FUS transducers are deployed in the treatment position; creating a tissue coupling interface if required; adjusting the volume of the coupling so as to position the focal point of the transducer correctly in the region to be treated; imaging the region of interest by scanning the imaging crystal over the region to be treated; delivering to a first location a dose of FUS that is distributed over a wide entrance angle by rotating the jaws while delivering FUS; moving the transducer to an at least second position by adjusting the shape of the flexible shaft under computer or manual control; delivering an additional dose of HIFU at the new position of the transducer; thereby scanning the focal spot of the transducer over the region to be treated and delivering a dose of thermal or cavitational energy to a region of tissue that is larger than the size of the focal spot of the transducer.

In embodiment of the present disclosure, an additional method of delivering a FUS treatment is provided. The method may include advancing into a patient through a surgically created or naturally occurring opening a probe that contains at least a single pair of closed jaws each fitted with a FUS transducer, that may contain an imaging transducer mounted at the center of the jaws, that includes a means for localizing at least the end of the probe relative to the target or to an external landmark, and includes a means for coupling the transducer to the tissue to be treated through which is installed an acoustic window; under computer or manual control advancing the probe while using a flexible drive shaft system that can change the shape of the probe so as to adjust the position of the end of the probe and bring it to the correct region to be treated as indicated by the localization device; opening the jaws under computer or manual control so that the FUS transducers are deployed in the treatment position; creating a tissue coupling interface if required; adjusting the volume of the coupling so as to position the focal point of the transducer correctly in the region to be treated; imaging the region of interest by scanning the imaging crystal over the region to be treated; delivering to a first location a dose of FUS that is distributed over a wide entrance angle by rotating the jaws while delivering FUS; moving the transducer under computer or manual control to an at least second position as defined by the localization system by adjusting the shape of the flexible shaft; delivering an additional dose of HIFU at the new position of the transducer; thereby scanning the focal spot of the transducer over the region to be treated and delivering a dose of thermal or cavitational energy to a region of tissue that is larger than the size of the focal spot of the transducer.

Another method of delivering a FUS treatment is provided according to a further embodiment of the present disclosure. The method may include advancing into a patient through a surgically created or naturally occurring opening a probe that contains at least a single pair of closed jaws each fitted with a FUS transducer, that may contain an imaging transducer mounted at the center of the jaws, that includes optical means for visualizing the region to be treated, and includes a means for coupling the transducer to the tissue to be treated through which is installed an acoustic window; under computer or manual control advancing the probe while using a flexible drive shaft system that can change the shape of the probe so as to adjust the position of the end of the probe and bring it in proximity to the region to be treated; confirming the correct location of the probe by optical visualization; opening the jaws under computer or manual control so that the FUS transducers are deployed in the treatment position; creating a tissue coupling interface if required; adjusting the volume of the coupling so as to position the focal point of the transducer correctly in the region to be treated; delivering to a first location a dose of FUS that is distributed over a wide entrance angle by rotating the jaws while delivering FUS; moving the transducer to an at least second position confirmed by the optical visualization system by adjusting the shape of the flexible shaft under computer or manual control; delivering an additional dose of HIFU at the new position of the transducer; thereby scanning the focal spot of the transducer over the region to be treated and delivering a dose of thermal or cavitational energy to a region of tissue that is larger than the size of the focal spot of the transducer.

A further method of delivering a FUS treatment is provided according to an embodiment of the present disclosure. The method may include advancing into a patient through a surgically created or naturally occurring opening a probe that contains at least a single pair of closed jaws each fitted with a FUS transducer, that may contain an imaging transducer mounted at the center of the jaws, that includes a means for localizing at least the end of the probe relative to the target or to an external landmark, that includes optical means for visualizing the region to be treated, and includes a means for coupling the transducer to the tissue to be treated through which is installed an acoustic window; under computer or manual control advancing the probe while using a flexible drive shaft system that can change the shape of the probe so as to adjust the position of the end of the probe and bring it to the correct region to be treated as indicated by the localization device; confirming the correct location of the probe by optical visualization; opening the jaws under computer or manual control so that the FUS transducers are deployed in the treatment position; creating a tissue coupling interface if required; adjusting the volume of the coupling so as to position the focal point of the transducer correctly in the region to be treated; imaging the region of interest by scanning the imaging crystal over the region to be treated; delivering to a first location a dose of FUS that is distributed over a wide entrance angle by rotating the jaws while delivering FUS; moving the transducer under computer or manual control to an at least second position as defined by the localization system and confirmed by the optical visualization system by adjusting the shape of the flexible shaft; delivering an additional dose of HIFU at the new position of the transducer; thereby scanning the focal spot of the transducer over the region to be treated and delivering a dose of thermal or cavitational energy to a region of tissue that is larger than the size of the focal spot of the transducer.

A further method of delivering a FUS treatment is provided according to an embodiment of the present disclosure. The method may include advancing into a patient through a surgically created or naturally occurring opening a probe, a probe that contains at least a single FUS transducer at its end aligned with the long axis of the probe; under computer or manual control advancing the probe while using a flexible drive shaft system that can change the shape and direction of the probe so as to direct the end of the probe to the correct region to be treated as indicated by a localization device or US or optical imaging system incorporated into the probe; rotating the end of the probe so as to direct the transducer to the region to be treated; creating a tissue coupling interface if required; adjusting the volume of the coupling so as to position the focal point of the transducer correctly in the region to be treated; delivering to a first location a dose of FUS; moving the transducer under computer or manual control to an at least second position by adjusting the shape of the flexible shaft and/or adjusting the position of the transducer relative to the shaft; delivering an additional dose of HIFU at the new position of the transducer; thereby scanning the focal spot of the transducer over the region to be treated and delivering a dose of thermal or cavitational energy to a region of tissue that is larger than the size of the focal spot of the transducer.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings various illustrative embodiments. It should be understood, however, that the invention is not limited to the precise arrangements and instrumentalities shown. In the drawings:

FIG. 1 is a schematic diagram of a system or probe assembly according to an exemplary embodiment of the present disclosure, wherein a pair of jaws is shown in a closed or compact configuration;

FIG. 2a is a schematic diagram of the probe assembly shown in FIG. 1, wherein the jaws are shown in an open or expanded configuration and controlled by jaw motor action;

FIG. 2b is a schematic diagram of a system or probe assembly according to an exemplary embodiment of the present disclosure, wherein a configuration of transducers may be controlled by one or more bend motors;

FIG. 2c is a schematic diagram of the probe shown in FIG. 2b, wherein a configuration of the transducers may be controlled by one or more shaft rotation motors;

FIG. 3a is an enlarged schematic diagram of a transducer arrangement of the probe shown in FIGS. 2b and 2c;

FIG. 3b is a schematic diagram of a transducer arrangement according to an exemplary embodiment of the present disclosure;

FIG. 4 is a schematic diagram of a system utilizing the probe assembly shown in FIG. 2b, wherein rotation of one or more transducers may create a distributed delivery angle for thermal dose;

FIG. 5a is a schematic diagram of a system or probe assembly according to an exemplary embodiment of the present disclosure, wherein a pair of transducers are shown in a closed or compact configuration;

FIG. 5b is a schematic diagram of the probe assembly shown in FIG. 5a, wherein the jaws are shown in an open or expanded configuration;

FIG. 5c is a schematic diagram of a system probe assembly according to an exemplary embodiment of the present disclosure, wherein at least one imaging transducer may emit one or more beams;

FIG. 5d is a schematic diagram of the probe assembly shown in FIG. 5c, wherein two therapy transducers may each emit one or more beams;

FIG. 5e is another schematic diagram of the probe assembly shown in FIGS. 5c and 5d;

FIG. 5f is a schematic of a system or probe assembly according the an exemplary embodiment of the present disclosure;

FIG. 5g is a schematic of a system or probe assembly according the an exemplary embodiment of the present disclosure;

FIG. 6a is a schematic diagram of a system or probe assembly according to an exemplary embodiment of the present disclosure;

FIG. 6b is a schematic diagram of the probe assembly shown in FIG. 6a, wherein transducers are oriented in a first direction; and

FIG. 6c is a schematic diagram of the probe assembly shown in FIG. 6a, wherein transducers are oriented in an opposing second direction.

### DETAILED DESCRIPTION

Various embodiments of the present disclosure are described hereinafter with reference to the figures. It should be noted that the figures are not drawn to scale and elements of similar structures or functions are represented by like reference numerals throughout the figures. It should also be noted that the figures are not intended to facilitate the description of specific embodiments of the invention. The figures are not intended as an exhaustive description of the invention or as a limitation on the scope of the invention. In addition, an aspect described in conjunction with a particular embodiment of the present disclosure is not necessarily limited to that embodiment and can be practiced in any other embodiments of the present disclosure. It will be appreciated that while various embodiments of the present disclosure are described in connection with radiation treatment of tumors, the claimed disclosure has application in other industries and to targets other than cancers.

Any headings used herein are for organizational purposes only and are not meant to limit the scope of the description or the claims. Certain terminology is used in the following description for convenience only and is not limiting. As used herein, the word “may” is used in a permissive sense (e.g., meaning having the potential to) rather than the mandatory sense (e.g., meaning must). Similarly, the words “a,” “an” and “the” mean “at least one,” and the words “include,” “includes” and “including” mean “including, but not limited to.”

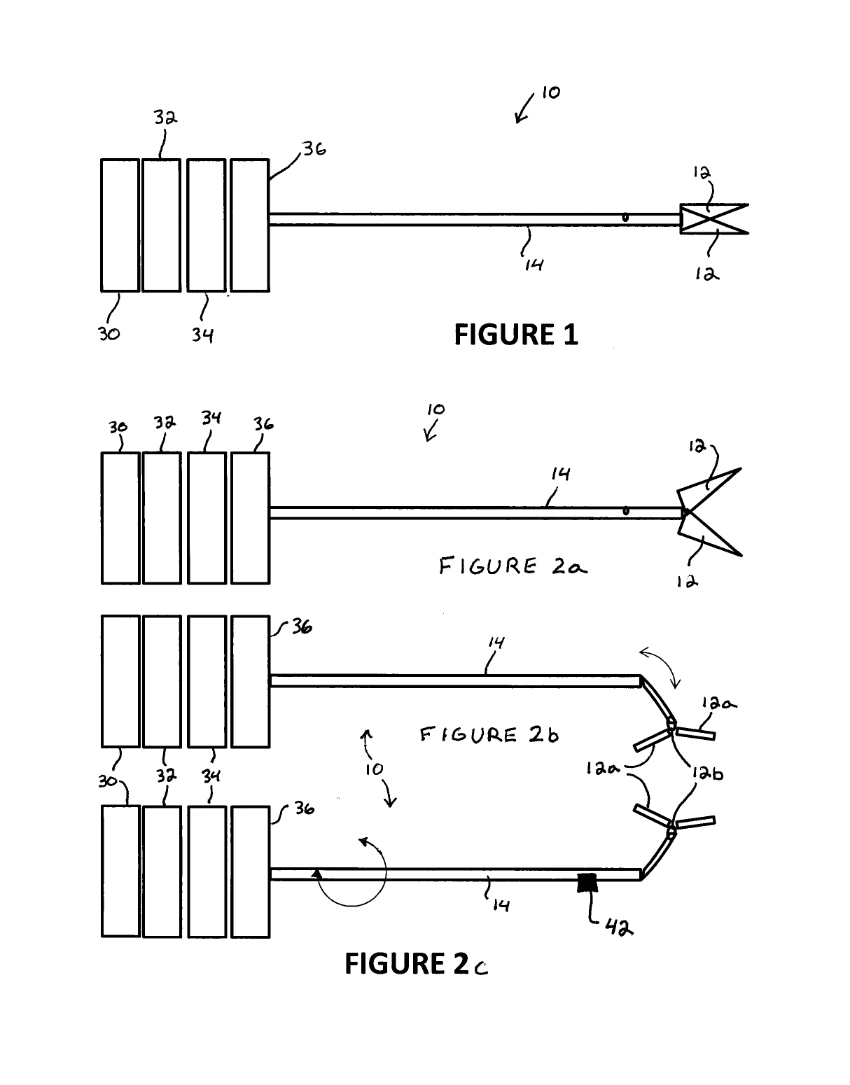

In one or more embodiments of the present disclosure, such as those depicted in FIGS. 1-6c, a system or probe, generally designated 10, may include one or more transducers 12 designed to deliver FUS. The transducer(s) 12 may be arranged as, or attached to, at least one or more movable elements or jaws positioned at and/or secured to or near a distal end 14a of a drive shaft 14. The drive shaft 14 may be at least generally flexible, such as at one or more discrete segments thereof or along an entire length of the drive shaft 14. The transducers 12 may include one or more therapy transducers 12a and/or one or more image transducers 12b. As understood by those skilled in the art, the therapy transducers 12a can include a fixed geometric focal spot or can include a focal spot that can be varied electronically, such as with an annular, linear, or phased array system.

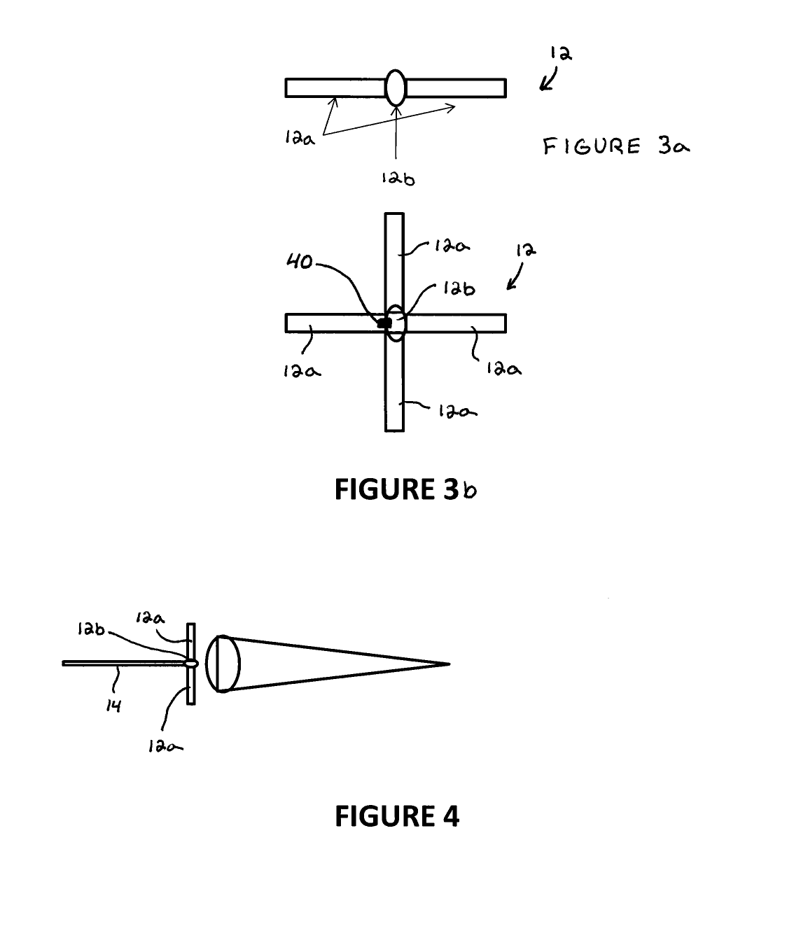

Referring to FIGS. 1-2c, the jaws may be connected through the drive shaft 14 to an apparatus or means for opening and closing the jaws in varying degrees under manual, motor and/or computer control. The apparatus or means for opening and closing the jaws may include one or more handles, levers, processors, motors or the like. For example, the probe 10 may include one or more jaw motors 30, one or more shaft rotation motors 32, one or more bend motors 34 and/or one or more tip rotation motors 36. Each of the motors 30, 32, 34, 36 may be positioned at or near a proximal end 14b of the drive shaft 14. Each of the motors 30, 32, 34, 36 may be separate and independent, or each of the motors 30, 32, 34, 36 may combine to form one, single motor.

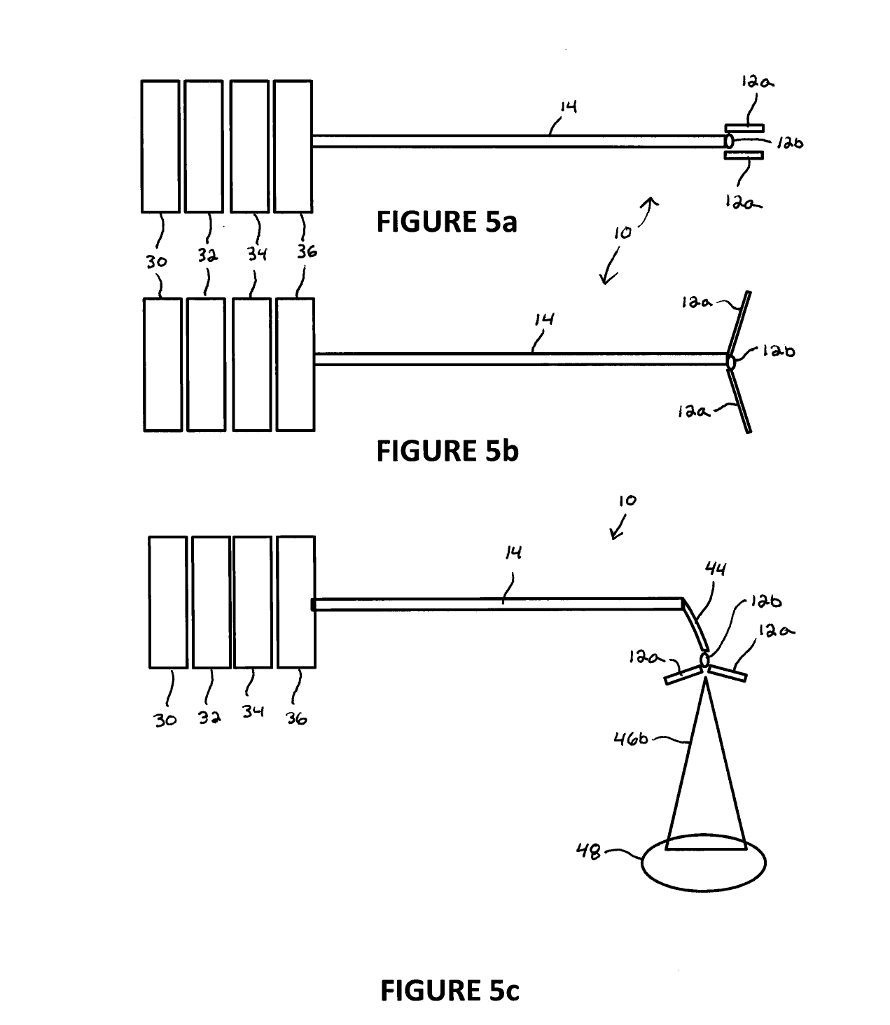

A point at which one or more lines passing perpendicularly to, and/or through a center of, a surface of each transducer 12 may determine the focal point of the transducers 12. Space allowing, additional pairs of jaws may be installed at or attached to the distal end 14a of the drive shaft 14 to create a rosette of transducers 12 (see FIG. 3b), which may include four or more therapy transducers 12a. In such an embodiment, all of the jaws may be opened and/or closed using the apparatus or another common mechanism. Thus, the drive shaft 14 can be activated under manual, motor and/or computer control to rotate the jaws through at least one hundred eighty degrees (180°). The drive shaft 14 may be placed inside a generally flexible second shaft whose shape can be adjusted under manual, motor and/or computer control using means well known to those skilled in the art. The second shaft can, in turn, be rotated through at least one hundred eighty degrees (180°) through a separate means secured to either the drive shaft 14 or to the motors or other means used to adjust the shape of the second shaft. As one non-limiting example, the drive shaft 14 and the second shaft may function similar to one tube inside another tube, wherein the outer tube (e.g., second shaft) protects the inner tube (e.g., drive shaft 14). The second shaft may be formed of any material that protects the drive shaft 14 from fluids and the like.

As shown in FIGS. 3a and 3b, one or more imaging US transducers 12b can be mounted at a the center of where the jaws are joined, thereby looking forward perpendicularly to an axis of opening of the jaws. Each imaging US transducer 12b can be a linear, annular, phased array or single crystal transducer. Wires running to the therapy and imaging transducers 12a, 12b can be run down or within the center of the drive shaft 12, for example. As shown in FIG. 3b, an optical imaging system 40, including a fiberoptic cable for delivering light to the end of the probe 10 and/or for sending images back to a camera system separate from the probe 10, also can be secured to or near the center of the jaw mechanism or can be run along an outside or inside of the flexible shafts terminating anywhere along the shafts. In a similar fashion, as shown in FIG. 2c, a magnetic localization system 42, possibly including a wire and/or a sensor, for example, can be mounted to the probe 10. The localization system 42 can be used to determine the position of at least a distal tip of the probe 10 using techniques known to those skilled in the art.

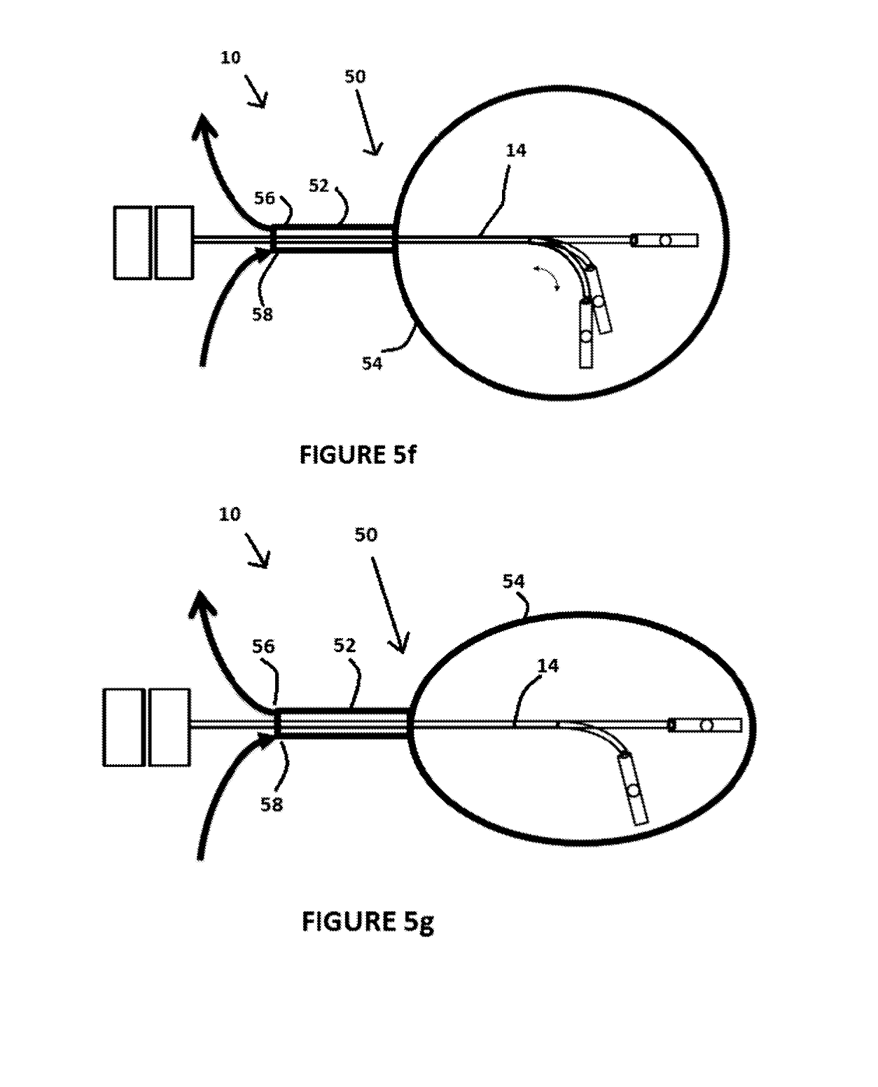

Referring to FIGS. 5f and 5g, a generally flexible and/or resilient fluid fillable membrane 50 may be secured to at least a portion of the probe 10 in a permanent or removable manner. The membrane 50 may include a non-distensible section 52 surrounding at least a portion of one or more of the shafts of the probe 10 and an inflatable portion 54 mounted at the distal end of the probe 10, such that at least a portion of the membrane 50 can be selectively enlarged in size by the injection of fluid to enclose and/or surround the jaws (e.g., transducers 12) when they are deployed in the treatment position. The membrane 50 may be equipped with ingress and egress ports 56, 58 at a proximal end thereof for controlling the flow of fluid therein and thereout. FIG. 5f shows the membrane 50 at an enlarged size, and FIG. 5g shows the membrane 50 in a contracted or reduced state. However, the membrane 50 is not limited to the size, shape and/or configurations shown and described herein.

The motor(s) 30, 32, 34, 36 that can be used to control the various states or configurations of the probe 10 can be housed at a proximal end thereof or can be mounted remotely at some distance from the probe 10 using long drive lines. The motor(s) 30, 32, 34, 36 can be activated manually and/or under computer control to alter the shape of the probe 10, the deployment of the transducers 12, and the rotation of the shafts and transducers 12. The motor(s) 30, 32, 34, 36 can be of types that can alter shape and position and orientation and deployment in discrete steps or continuously. The motor(s) 30, 32, 34, 36 also can be replaced by manual means for adjusting shape and position and orientation and deployment.

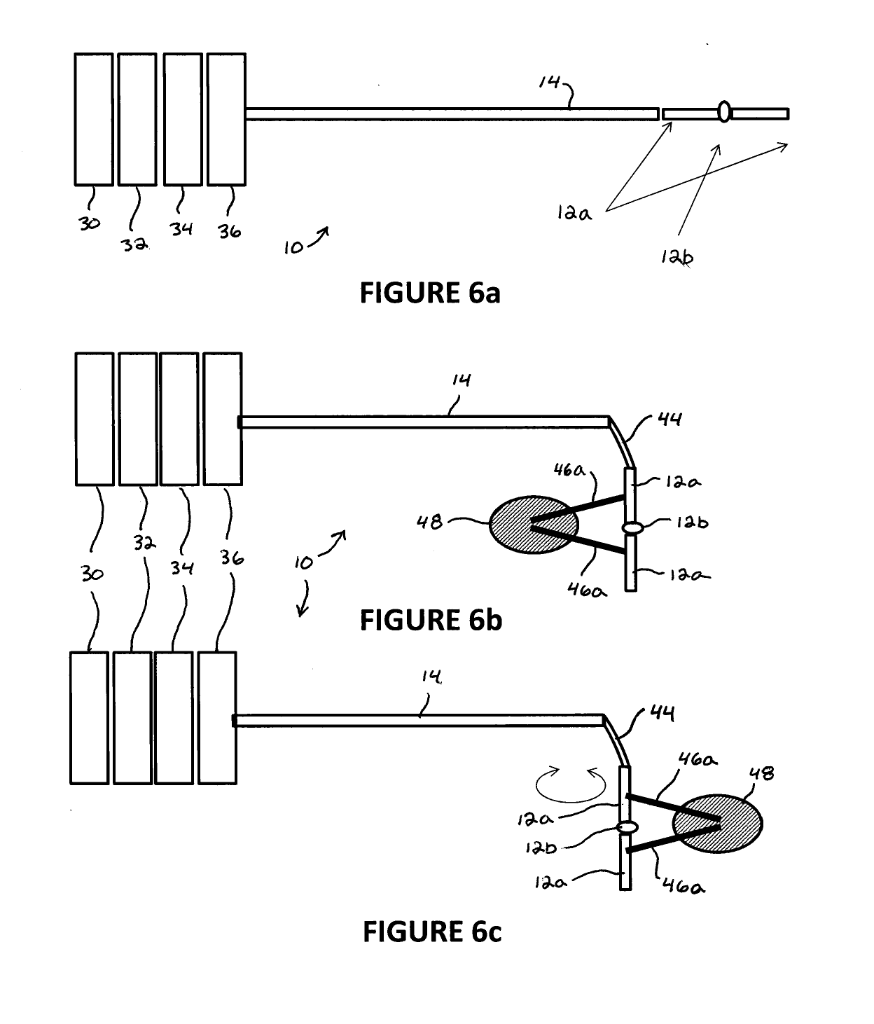

Referring to FIGS. 6a-6c, in another embodiment of the present disclosure, instead of securing the transducer(s) 12 to jaws mounted on the end of the flexible drive shaft 14, the transducers 12 can be mounted to the drive shaft 14 in an orientation such that one or more therapeutic beams 46a generated by the transducers 12 are at least generally orthogonal to a longitudinal axis of the drive shaft 14. The mounting may be accomplished with one or more rotational and/or flexible joints 44, such that at least the active portion of the transducers 12 can be rotated relative to the drive shaft 14. The joint(s) 44 may be any device that allows one portion of the drive shaft 14 to be rotated with respect to another portion of the drive shaft 14 and/or the probe 10. The joint(s) 44 can be secured to a means for moving the transducers 12 linearly relative to the drive shaft 14. The joint(s) 44 may be part of or integral with the drive shaft 14, or the drive shaft 14 and the joint(s) 44 may be separate or independent components. In one embodiment, at least a portion of the joint 44 may be selectively retractable within and/or extendable from an interior of the drive shaft 14.

In operation, the probe 10 may be inserted at least partially through a naturally occurring opening in a patient, such as the rectum, the urethra, the mouth or the nasal passage, for example, or through a surgically created opening. Referring to FIGS. 1, 5a and 6a, during insertion, the jaws may be at least partially or completed closed (see FIGS. 1 and 5a) or the transducer(s) 12 may be in a linear configuration (see FIG. 6a). Under visualization provided either by an ancillary means of optic imaging, such as an endoscope, by optical imaging provided by the probe 10 if it is so equipped, by US imaging provided by the probe 10 if it is so equipped, by radiological means of imaging including x-rays, MRI, and other 3-D volumetric means of imaging, and/or by a magnetic localization system if the probe 10 is so equipped, the probe 10 can be guided to the correct treatment location through a combination of advancing the probe 10 and adjusting the shape of at least one or more portions of the probe 10 by manual and/or computer-controlled means. The same control means can be used to position the distal end of the probe 10 the correct or appropriate distance from the tissue to be treated so that the focal point of the transducers 12, once deployed, is located at least partially or completely inside the tissue to be treated.

Once the probe 10 is in the correct or appropriate location, the membrane may be partially or completely filled with fluid, the distal end of the membrane may be enlarged by an amount proportional to the amount of fluid instilled in the membrane, thereby positioning the distal end of the probe 10 a defined distance from the tissue to be treated. As shown in FIG. 5b, the mechanism for opening the jaws, such as the motor(s) 30, 32, 34, 36, may be activated with the desired amount of jaw opening being determined by the focal distance to be utilized in the treatment.

As shown in FIG. 5c, the various means for imaging can be used to confirm the correct placement of the therapy transducers 12a relative to the target tissue 48 (e.g., one or more tumors, lesions and the like) and then the treatment can be initiated. Referring to FIG. 5d, once the required amount of energy is delivered to tissue at the focal point of the transducers 12, the position of the focal point can be adjusted, if required, by adjusting the shape of one or more of the shafts 12 or the angulation of the shafts 12, in order to deliver heat to additional regions of tissue (see FIG. 5e) if the region to be treated is greater than the volume of tissue treated at the first focal spot. The adjustment can be made in discrete steps, whereby the FUS energy dwells on a volume of tissue for a fixed amount of time, is turned off, moved to a new location and reactivated, can occur in a continuous fashion, whereby the energy is kept on while the focal spot is moved at a predetermined speed, or can occur using a combination of the two approaches. Each subsequent position of the therapy transducers 12a can be confirmed by the various means for imaging described previously, and each position can be guided and achieved automatically under computer control according to a predetermined and planned pattern of therapy delivery.

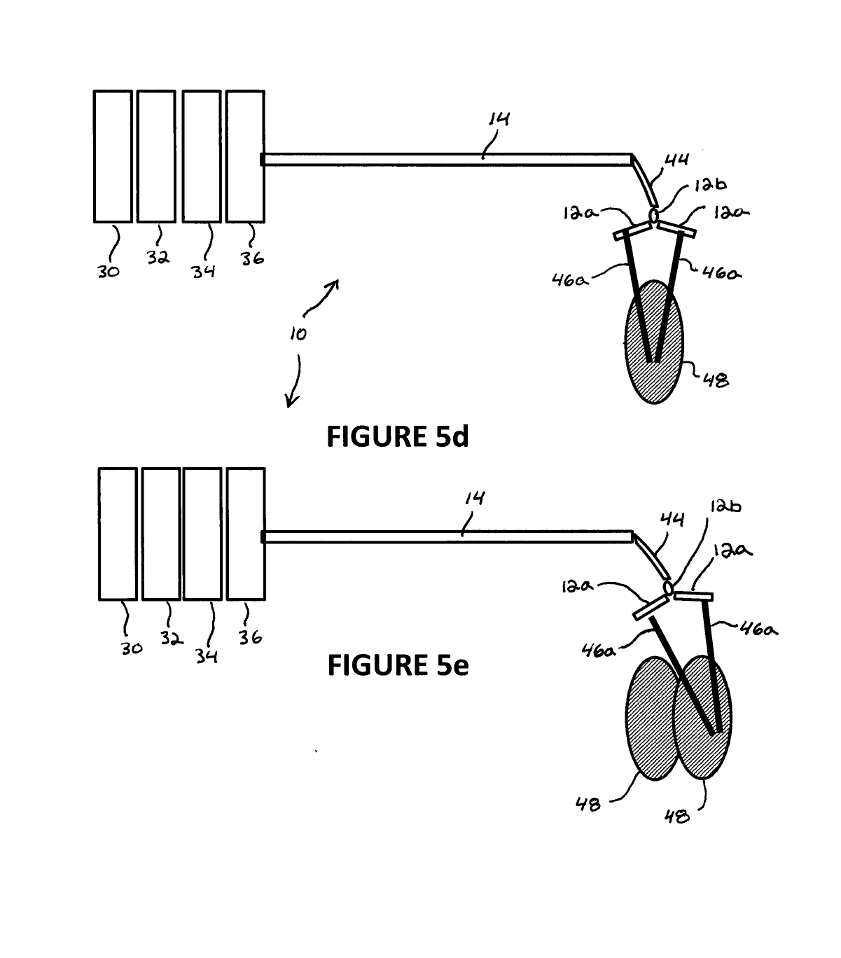

Alternatively, referring to FIG. 4, simultaneous with activating the therapy transducers 12a at each treatment position, the jaws can be rotated through as many as one hundred eighty degrees (180°). Such movement distributes the energy delivered to the focal point over an increased entrance angle, thereby minimizing the amount of energy, and the heat, received by tissue between the transducers 12 and the focal point.

When employing an imaging transducer 12b that incorporates some form of array, the region to be imaged can be scanned by the transducer 12 directly. Where a single fixed crystal is employed, the crystal may be scanned mechanically over the region to be imaged. This can be done, while the imaging is activated, by adjusting in a continuous or stepwise fashion the shape and orientation of the flexible drive shaft 14 so that the imaging beam 46b is swept over the region of interest. Line data generated from each effective position of the imaging crystal can be compiled to generate 2-D or volumetric representations of the region. Positional information can be gathered for each position of the imaging crystal by the use of a magnetic localization device affixed to the end of the drive shaft 14 or by encoders affixed to the motors controlling the shape and orientation of the drive shaft 14.

In the example where the transducers 12 are mounted to a rotational means secured to the distal end 14a of the drive shaft 14 rather than as jaws (see, for example, FIG. 6a), the correct position of the transducers 12 relative to the tissue to be treated may be achieved by rotating the transducers 12 relative to the drive shaft 14. For instance, as shown in FIGS. 6b and 6c, with the drive shaft 14 and/or the joint 44 shaped so that it forms at least approximately or exactly a ninety degree (90°) bend, one or more beams 46a, 46b generated by the therapy and imaging transducers 12a, 12b can be directed up or down or right or left by rotating the end of the drive shaft 14. If the transducers 12 connection also has the capability to move the transducer(s) 12 in a linear fashion, doing so during imaging and/or therapy will provide an additional means for scanning the dose in addition to other means described above.

It will be appreciated by those skilled in the art that changes could be made to the embodiments described above without departing from the broad inventive concept thereof. It is understood, therefore, that this disclosure is not limited to the particular embodiments disclosed, but it is intended to cover modifications within the spirit and scope of the present disclosure as defined by the appended claims.

I/We claim:

1. A system for providing ultrasound, the system comprising:
a drive shaft having a proximal end and a distal end;
one or more motors positioned at or near the proximal end of the drive shaft;
one or more pair of jaws mounted on or near the distal end of the drive shaft; and
one or more transducers configured to generate thermal or cavitational lesions with ultrasound, each transducer being mounted to one of the jaws.

2. The system to claim 1, wherein the one or more transducers are configured to image the lesions with ultrasound.

3. The system according to claim 1, further comprising:
a tissue coupling mechanism including a fixed or variable fillable fluid membrane with ingress and egress ports, wherein the tissue coupling mechanism at least partially covers the one or more transducers.

4. The system according to claim 1, wherein the one or more motors are configured to be activated manually or under computer control to adjust the position of the one or more transducers.

5. The system according to claim 4, wherein the one or more motors are configured to control at least one of bending of the drive shaft, opening and closing of the pair of jaws, rotation of the shaft and the pair of jaws together, and rotation of the pair of jaws.

6. The system according to claim 1, wherein one or more beam emanating from the one or more transducers extend perpendicularly to a longitudinal axis of the drive shaft.

7. The system according to claim 1, wherein the one or more transducers include two therapy transducers and one image transducer.

8. The system according to claim 1, wherein the one or more motors includes a jaw motor, a shaft rotation motor, a bend motor and a tip rotation motor.

9. The system according to claim 1, wherein the pair of jaws is movable between an open position and a closed position.

10. A system for providing ultrasound, the system comprising:
a drive shaft having a proximal end and a distal end;
one or more motors positioned at or near the proximal end of the drive shaft;
at least one joint mounted to or near the distal end of the drive shaft; and
one or more transducers configured to generate thermal or cavitational lesions with ultrasound, each transducer being operatively connected to the at least one joint.

11. The system according to claim 10, wherein the one or more transducers include at least two therapy transducers and one image transducer, and wherein the image transducer is positioned between the two therapy transducers.

12. The system according to claim 10, wherein the at least two therapy transducers include a rosette of therapy transducers.

13. The system according to claim 10, wherein the one or more motors are configured to control at least one of bending of the drive shaft, opening and closing of the pair of jaws, rotation of the shaft and the pair of jaws together, and rotation of the pair of jaws.

14. A method for providing ultrasound, the method comprising:
advancing at least a portion of a probe through a surgically created or naturally occurring opening in a patient, the probe including at least a flexible drive shaft and a pair of jaws having at least one imaging transducer, the jaws being in a closed configuration;
coupling the transducer to tissue of the patient;
changing the shape of the probe via the flexible drive shaft;
opening the jaws; and
imaging a region of tissue of the patient by scanning the transducer over the rejoin.

15. The method according to claim 14, wherein the at least one imagining transducer is mounted at a center of the pair of jaws.

16. The method according to claim 14, further comprising:
delivering focused ultrasound to a first location while rotating the jaws.

17. The method according to claim 16, further comprising:
moving the transducer by adjusting the shape of the flexible drive shaft; and
delivering another dose of focused ultrasound to another region of tissue of the patient.

18. The method according to claim 14, further comprising:
partially or completely filing a membrane once the probe is in a correct or appropriate location, thereby positioning the distal end of the probe a defined distance from the region of tissue.

19. The method according to claim 14, wherein a degree to which the jaws are opened is determined by a focal distance to be utilized in treatment.

20. The method according to claim 14, further comprising:
rotating the jaws of the probe while simultaneous activating therapy transducers.
